(19) United States
(12) Patent Application Publication
(10) Pub. No.: US 2005/0171555 A1
(43) Pub. Date: Aug. 4, 2005

(54) Intraocular lens injector

(76) Inventors: Son Trung Tran, Arlington, TX (US); Dyson W. Hickingbotham, Stouchsburg, PA (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/768,948

(22) Filed: Jan. 30, 2004

(51) Int. Cl. A61F 9/00

(52) U.S. Cl. 606/107

(57) ABSTRACT

A device having a pair of stretcher bars. The first bar remains fixed while the second bar can be reciprocated relative to the first bar by actuation of a movable slide. The device of the present invention is useful for stretching and implanting the ring portion of a two component intraocular lens.

This invention relates to intraocular lenses (IOLs) and more particularly to injectors used to inject IOLs into an eye.

### BACKGROUND OF THE INVENTION

The human eye in its simplest terms functions to provide vision by transmitting and refracting light through a clear outer portion called the cornea, and further focusing the image by way of the lens onto the retina at the back of the eye. The quality of the focused image depends on many factors including the size, shape and length of the eye, and the shape and transparency of the cornea and lens.

When trauma, age or disease cause the lens to become less transparent, vision deteriorates because of the diminished light which can be transmitted to the retina. This deficiency in the lens of the eye is medically known as a cataract. The treatment for this condition is surgical removal of the lens and implantation of an artificial lens or IOL.

While early IOLs were made from hard plastic, such as polymethylmethacrylate (PMMA), soft, foldable IOLs made from silicone, soft acrylics and hydrogels have become increasingly popular because of the ability to fold or roll these soft lenses and insert them through a smaller incision. Several methods of rolling or folding the lenses are used. One popular method is an injector cartridge that folds the lenses and provides a relatively small diameter lumen through which the lens may be pushed into the eye, usually by a soft tip plunger. The most commonly used injector cartridge design is illustrated in U.S. Pat. No. 4,681,102 (Bartell), and includes a split, longitudinally hinged cartridge. Similar designs are illustrated in U.S. Pat. Nos. 5,494,484 and 5,499,987 (Feingold) and U.S. Pat. Nos. 5,616,148 and 5,620,450 (Eagles, et al.). In an attempt to avoid the claims of U.S. Pat. No. 4,681,102, several solid cartridges have been investigated, see for example U.S. Pat. No. 5,275,604 (Rheinish, et al.) and U.S. Pat. No. 5,653,715 (Reich, et al.).

These prior art devices used plungers that were substantially symmetrical about a longitudinal axis and generally contained a cylindrical or flared soft tip that completely filled the bore of the injector cartridge. See, for example, U.S. Pat. No. 4,681,102 (Bartell) and U.S. Pat. No. 4,919,130 (Stoy, et al.), and WIPO Publication No. WO 96/29956, the entire contents of which are incorporated herein by reference. Other plungers had hooked or forked tips meant to grasp the edge of the IOL. See for example, U.S. Pat. No. 4,573,998 (Mazzocco), U.S. Pat. Nos. 5,494,484 and 5,499,484 (Feingold), U.S. Pat. Nos. 5,616,148 and 5,620,450 (Eagles, et al.) and U.S. Pat. No. 5,653,715 (Reich, et al.), the entire contents of which are incorporated herein by reference. One plunger tip recently commercially introduced is designed so that the IOL rolls around the tip as the IOL is advanced down the cartridge. See U.S. Pat. No. 5,735,858 (Makker, et al.), the entire contents of which is incorporated herein by reference.

While these symmetric plunger designs work well, the large tip requires a relatively large incision, usually on the order of 3.0 mm or larger. Accordingly, a need continues to exist for an IOL injector designed to be used with incisions smaller than 3.0 mm.

### BRIEF SUMMARY OF THE INVENTION

The present invention improves upon prior art lens injectors by providing a device having a pair of stretcher bars. The first bar remains fixed while the second bar can be reciprocated relative to the first bar by actuation of a movable slide. The device of the present invention is useful for stretching and implanting the ring portion of a two component intraocular lens.

It is accordingly an object of the present invention to provide a lens injector having a pair of stretcher bars.

It is a further object of the present invention to provide a lens injector having a pair of stretcher bars that move relative to each other.

It is a further object of the present invention to provide a lens injector useful for stretching and implanting the ring portion of a two component intraocular lens.

Other objects, features and advantages of the present invention will become apparent with reference to the drawings, and the following description of the drawings and claims.

### BRIEF DESCRIPTION OF THE DRAWINGS

FIG. 1 is an enlarged perspective view of the lens injector of the present invention.

FIG. 2a is an enlarged perspective view of a first embodiment of the tip of the lens injector of the present invention taken at circle 2 in FIG. 1, showing the stretcher bars in the relaxed position.

FIG. 2b is an enlarged perspective view of a first embodiment of the tip of the lens injector of the present invention taken at circle 2 in FIG. 1, showing the stretcher bars in the retracted position.

FIG. 3 is a side elevational view of a second embodiment of the tip of the lens injector of the present invention.

FIG. 4 is a bottom plan view of a second embodiment of the tip of the lens injector of the present invention.

FIG. 5 is a side elevational view of a third embodiment of the tip of the lens injector of the present invention.

FIG. 6 is a bottom plan view of a third embodiment of the tip of the lens injector of the present invention.

FIG. 7 is a perspective view of a third embodiment of the tip of the lens injector of the present invention.

### DETAILED DESCRIPTION OF THE INVENTION

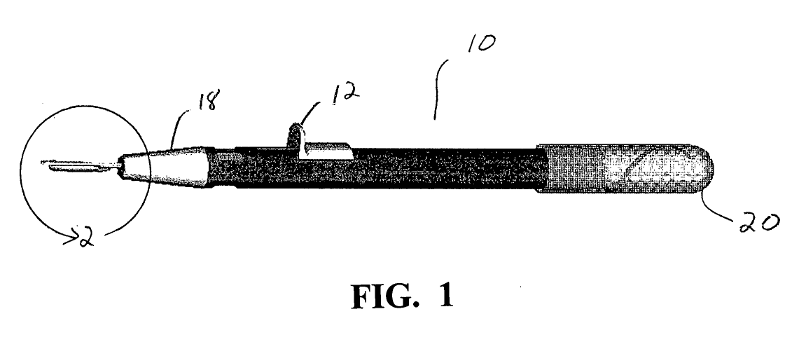

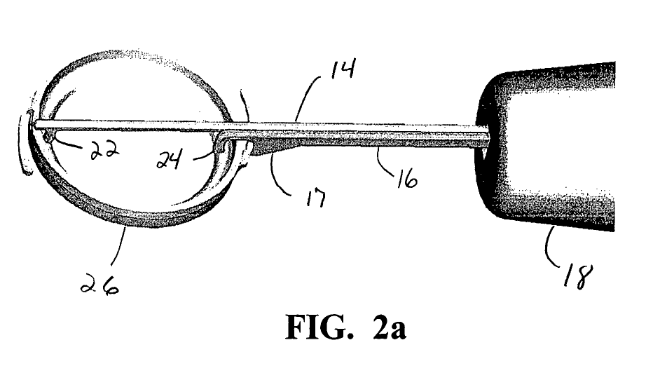

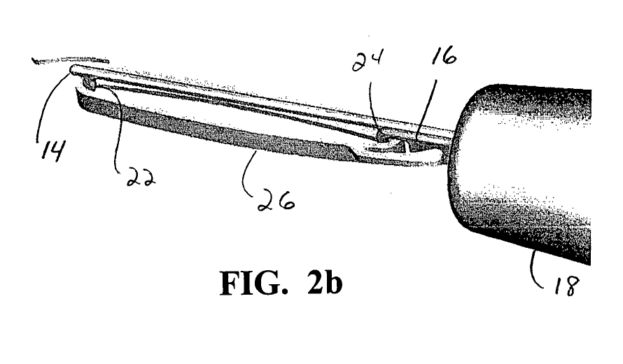

As best seen in FIGS. 1, 2a and 2b, intraocular lens injector 10 of the present invention generally consist of handpiece 10 and stretcher bars 14 and 16. Handpiece 10 contains reciprocating thumb slide 12, nosepiece 18 and extendable grip 20. Grip 20 may be extended or retracted so as to fit the hand of the user more comfortable. Stretcher bar 14 is fixed to nosepiece 18 while stretcher bar 16 penetrates through nosepiece 18 and is fixed to thumb slide 12, so that reciprocation of thumb slide 12 causes corresponding reciprocation of stretcher bar 16 relative to stretcher bar 14. Thumb slide 12 may be spring biased in either direction and may be operated by means other than manual manipulation, such as electrically or pneumatically. Stretcher bar 14 contains hook 22 and stretcher bar 16 contains hook 24. Stretcher bars 14 and 16 are preferably made from titanium, stainless steel or thermoplastic.

As best seen in FIGS. 2a and 2b, intraocular lens injector 10 is best used in combination with ring component 26 of a multicomponent lens. One suitable lens is described in U.S. patent application Ser. No. 10/618,954, the entire contents of which being incorporated herein by reference and specifically, the description of the lens system on page 3, lines 25-28 and on page 4, lines 1-14 and FIGS. 1, 2 and 3. Ring component 26 is held in place in hooks 22 and 24 on stretcher bars 14 and 16, respectively. Movement of thumb slide 12 causes hook 24 to move toward or away from hook 22, depending upon the direction of movement of thumb slide 12. Moving hook 24 away from hook 22 cause ring component 26 to stretch, as seen in FIG. 2b. Stretching of ring component 26 causes ring component 26 to become thinner in cross-sectional area. When stretched, ring component 26 may be inserted into an eye through a relatively incision smaller incision, preferably less than 3 mm and more preferably, 2.00 mm or less. As best seen in FIG. 2a, stretcher bar 16 contains projection 17. Projection 17 helps prevent hook 24 from catching on the wound when injector 10 is removed from an eye.

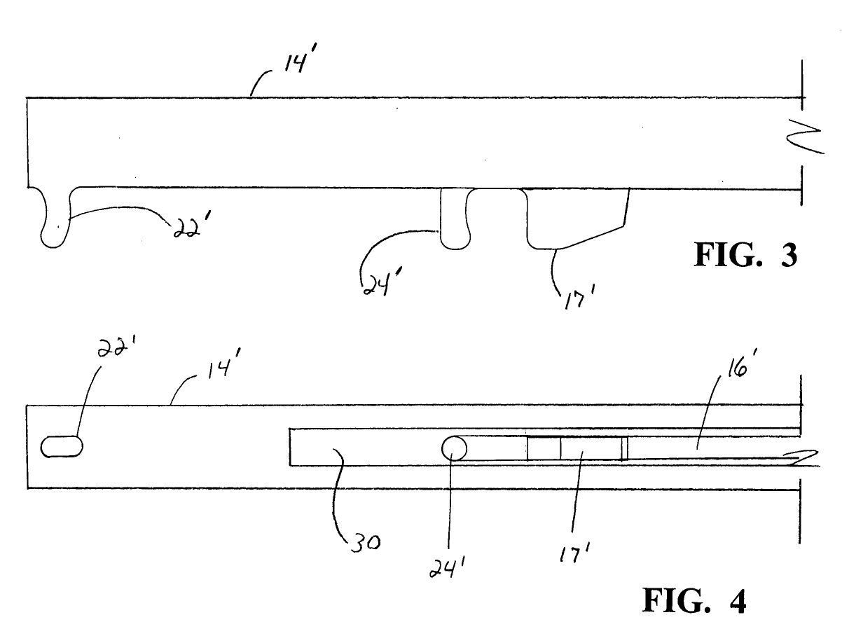

As best seen in FIGS. 3 and 4, stretcher bar 14′ may alternatively consist of a hollow tube surrounding stretcher bar 16′ so that stretcher bars 14′ and 16′ are coaxial, and stretcher bar 16′ reciprocates within stretcher bars 14′ in slot 30 so as to move hooks 22′ and 24′ closer or father apart. Stretcher bar 16′ also contains projection 17′.

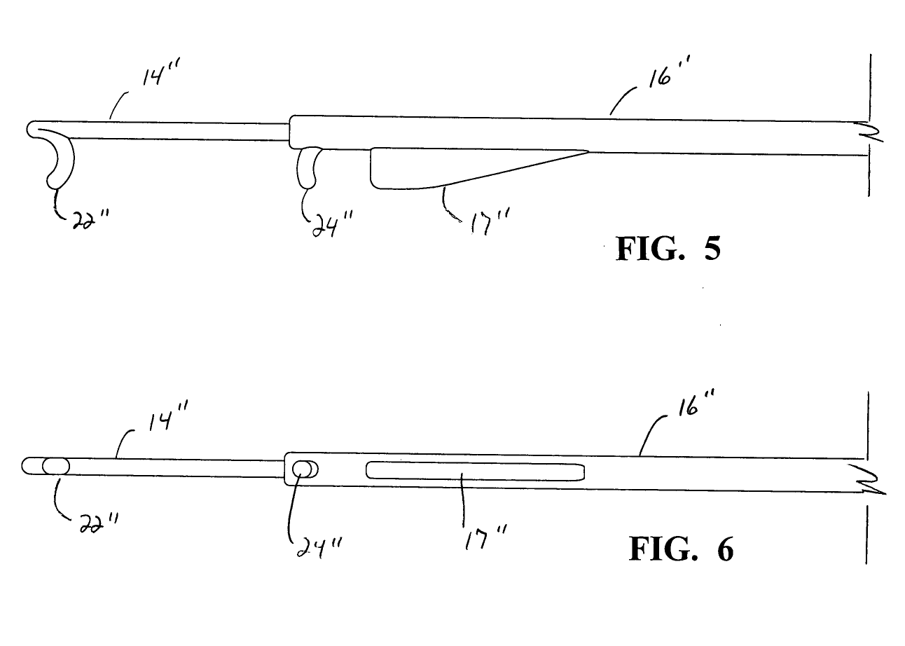

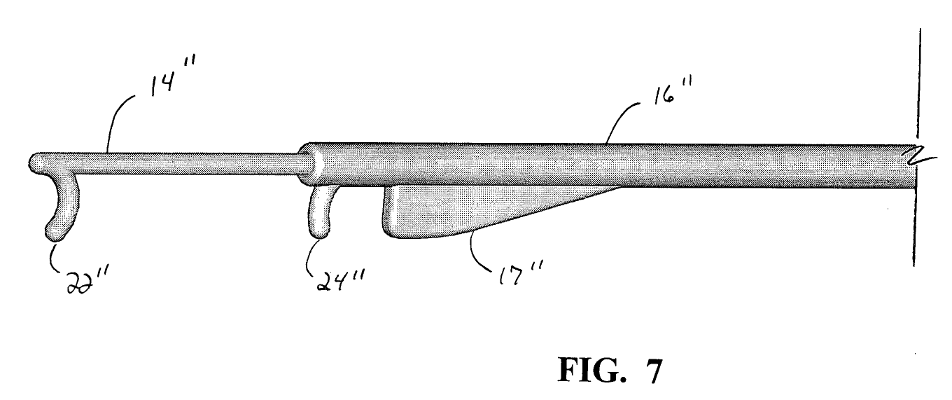

As best seen in FIGS. 5, 6 and 7, stretcher bar 14″ may project out the distal tip of stretcher bar 16″. In such a construction, stretcher bar 14″ may be fixed so that stretcher bar 16″ reciprocates coaxially over stretcher bar 14″. Stretcher bar 14″ contains hook 22″ and stretcher bar 16″ contains hook 24″ and projection 17″ and is otherwise of construction similar to that described above.

While certain embodiments of the present invention have been described above, these descriptions are given for purposes of illustration and explanation. Variations, changes, modifications and departures from the systems and methods disclosed above may be adopted without departure from the scope or spirit of the present invention.

1. An intraocular lens injector system, comprising:
a) a handpiece having a nosepiece;
b) a first stretcher bar fixed to the nosepiece and projecting from the handpiece; and
c) a second stretcher bar penetrating the nosepiece, projecting from the handpiece and attached to the handpiece so as to allow the second stretcher bar to reciprocate relative to the first stretcher bar.

2. The lens injector system of claim 1 wherein the stretcher bars contain hooks.

3. The lens injector system of claim 1 wherein the first and second stretcher bars are coaxial.

4. The lens injector system of claim 1 wherein the second stretcher bar is mounted to a thumb slide on the handpiece.

5. An intraocular lens injector system, comprising:
a) a handpiece having a nosepiece;
b) a first stretcher bar having a first hook; and
c) a second stretcher bar having a second hook, the second stretcher bar being coaxially mounted within the first stretcher bar,
wherein the first and the second stretcher bars are attached to the handpiece so as to allow the first and the second stretcher bars to reciprocate relative to each other.

6. The lens injector system of claim 5 wherein the second stretcher bar is mounted to a thumb slide on the handpiece.

7. The lens injector system of claim 5 wherein the first stretcher bar is mounted to a thumb slide on the handpiece.
